(19) United States
(12) Patent Application Publication
(10) Pub. No.: US 2025/0352575 A1
(43) Pub. Date: Nov. 20, 2025

(54) MANUFACTURING AND APPLICATION OF MANGANESE-BASED THERANOSTIC NANOPARTICLE TECHNOLOGY

(71) Applicant: THE GOVERNING COUNCIL OF THE UNIVERSITY OF TORONTO, Toronto (CA)

(72) Inventors: Xiaoyu Wu, Toronto, Ontario (CA); Azhar Abasi, Toronto, Ontario, CA (US); Chunsheng He, Woodbridge, Ontario (CA); Mohammad Amini, Toronto, Ontario (CA); Tin-yo Yen, Toronto, Ontario (CA)

(21) Appl. No.: 18/874,435

(22) PCT Filed: Jul. 13, 2023
(86) PCT No.: PCT/CA2023/050943

§ 371 Date: Dec. 12, 2024

Related U.S. Application Data
(60) Provisional application No. 63/388,967, filed on Jul. 13, 2022.

(51) Int. Cl. A61K 33/24; A61K 9/51; A61K 47/69; A61K 49/18; A61P 35/00

(52) U.S. Cl. CPC A61K 33/24; A61K 9/5123; A61K 47/6923; A61K 49/1827; A61P 35/00

(57) ABSTRACT

A method of manufacture and compositions and uses of a multifunctional bioinorganic theranostic nanoconstruct are disclosed. Nanoconstructs described herein contain manganese dioxide in a biocompatible matrix and are useful for, e.g., MRI contrast imaging of tumor environments and enhancement of radiation therapy in cancer. Some nanoconstructs described herein incorporate targeting agents for specific targeting of cancer cells.

### BACKGROUND

Radiation therapy (RT) is a major treatment modality for cancer, including glioblastoma (GBM). However, hypoxic tumor microenvironment (TME)-mediated radioresistance among other factors has led to poor treatment responses. Although various approaches have been investigated to sensitize RT, the outcomes are disappointing in clinical trials due largely to ineffective delivery of molecular O₂ or radiation sensitizers into tumors. While a concern in breast, prostate and other tumor types, ineffective delivery is particularly relevant to GBM tumor masses because of older therapies' poor penetration across the blood-brain barrier (BBB) and blood-tumor barrier (BTB).

In addition, monitoring of molecular O₂ or radiation sensitizer delivery is often challenging, which heightens uncertainty as to extent of delivery to the tumor. There remains a need to develop trackable approaches to improve radiation therapy outcomes in breast, prostate, GBM, as well as in other cancer or tumor types.

### SUMMARY

Disclosed herein is a novel, robust method for the synthesis and scale-up manufacturing of pharmaceutically acceptable multifunctional and colloidally stable bioinorganic multifunctional theranostic nanoconstruct (NCs). NCs disclosed herein are constructed of MnO₂ nanoparticles loaded into a biocompatible matrix (thus forming an emulsion) and optionally coated with one or more additional layers. Implementations may include one or more of the following. The added manganese dioxide is provided as precursor nanoparticles approximately 5 nanometers to approximately 80 nanometers in size. The emulsion is processed through a high-pressure homogenizer. The NC is concentrated, optionally by using tangential flow filtration. The NC is lyophilized, optionally in the presence of a cryoprotectant. The biocompatible matrix is polymeric. The biocompatible matrix is lipidic. The biocompatible matrix is selected from polyelectrolyte-lipid, polyvinyl alcohol/lipid complex, phospholipids, graft terpolymer, poly(methacrylic acid)-polysorbate 80-starch (TERP), and fatty acids. The biocompatible matrix may also contain cholesterol. The additional layer comprises TERP.

Also disclosed herein are compositions and methods that include NCs as described above having an additional layer, where the additional layer is complexed with a targeting agent for the treatment of a cancer. Implementations may include one or more of the following. The cancer is prostate cancer and the targeting agent targets PSMA. The cancer is GBM and the targeting agent targets PSMA. The cancer is pancreatic cancer. The cancer is breast cancer. The targeting agent is TERP functionalized with Glu-urea-Lys. Methods include enhancement of cancer radiation therapy via injecting the subject with an NC described herein shortly prior to receiving radiation therapy.

Among other uses, NCs disclosed herein are capable of enhancing RT and/or magnetic resonance imaging (MRI) of tumors, including in GBM due to the ability of NCs disclosed herein to cross the BBB and BTB. Implementations may include one or more of the following. NCs are loaded with a magnetically resonant material. The magnetically resonant material is gadolinium, manganese, iron, or an oxide thereof.

These and other features and aspects, and combinations of them, may be expressed as methods, systems, components, means and steps for performing functions, apparatus, articles of manufacture, compositions of matter, and in other ways.

Among other advantages, methods disclosed herein are batch-to-batch consistent, reproducible, and easily scalable to large scale synthesis of an NC with physiological properties suitable for intravenous (IV) injection. The manufacturing process can produce NC suspensions at liter scale. The synthesis conditions can be optimized by using different lipids, adjusting the pressure and cycles of high-pressure homogenizer to obtain stable suspensions of small size NCs, with a narrow polydispersity index (PDI) that is suitable for intravenous injection.

In accord with the invention, there is provided a method of manufacture of a bioinorganic multifunctional theranostic nanoconstruct, the method comprising: adding manganese dioxide to a biocompatible matrix, thereby producing an emulsion of MnO₂ nanoparticles coated with the matrix; adding an additional layer to the emulsion; and processing the emulsion through a high-pressure homogenizer, thereby manufacturing the bioinorganic multifunctional theranostic nanoconstruct.

In an aspect of this invention, the method further comprises concentrating the nanoconstruct. In another aspect, the concentrating is performed using tangential flow filtration. In another aspect, the method further comprises lyophilizing the concentrated nanoconstruct. In still another aspect, the lyophilizing takes place in the presence of a cryoprotectant. In still another aspect, the added manganese dioxide are precursor nanoparticles approximately 5 nanometers to approximately 80 nanometers in size. In still another aspect, the biocompatible matrix is lipid. In yet another aspect, the biocompatible matrix is polymeric. In another aspect, the biocompatible matrix is selected from polyelectrolyte-lipid, polyvinyl alcohol/lipid complex, graft TERP, phospholipids, poly(methacrylic acid)-polysorbate 80-starch (TERP), and fatty acids, optionally also including cholesterol. In another aspect, the additional layer comprises TERP.

In accord with the invention, there is provided a bioinorganic multifunctional theranostic nanoconstruct comprising nanoscale manganese dioxide nanoparticles situated in a lipid emulsion, where the nanoparticles are coated with the lipid and further coated with a TERP functionalized with a targeting agent for cancer therapy. In an aspect of the invention, the therapeutic agent targets PSMA and the functionalized TERP is TERP-Glu-urea-Lys.

In accord with the invention, there is provided a bioinorganic multifunctional theranostic nanoconstruct comprising nanoscale manganese dioxide emulsified with lipid and further coated with TERP, wherein the nanoconstruct is loaded with a magnetically resonant material for use in MRI imaging. In an aspect of the invention, the magnetically resonant material is selected from gadolinium, manganese, iron, and oxides thereof.

In accord with the invention, there is provided a method of enhancing radiation therapy in a subject having a cancerous tumor, the method comprising, shortly prior to receiving radiation therapy, injecting the subject with a nanoconstruct of any of the preceding claims. In an aspect of the invention, the cancerous tumor is prostate cancer. In another aspect, the cancerous tumor is glioblastoma. In another aspect, the cancerous tumor is pancreatic cancer. In still another aspect, the cancerous tumor is breast cancer.

Other advantages and features will become apparent from the following description and claims.

### DESCRIPTION OF THE FIGURES

FIG. 1 illustrates the physical characteristics of an NC described herein.

FIG. 2 displays the ultraviolet-visible spectrum of an NC described herein.

FIG. 3 displays the thermal stability of an NC described herein using differential scanning calorimetry.

FIG. 4 illustrates an NC reaction rate up to 60 mins at H₂O₂ concentrations of 5 (top), 50 (middle), and 500 (bottom) micromolar.

FIG. 5 displays the MRI relaxivity of an NC described herein.

FIG. 6 shows the cytotoxicity of an NC described herein when tested on human breast epithelial cells.

FIG. 7 shows uptake and cancer cell viability of an NC described herein when tested on MDA-MB-231 breast cancer, PC3 prostate cancer and U87 brain cancer cells following 4 hr incubation with fluorescently-labeled NC.

FIG. 8 shows ICP-AES analysis of manganese following first and fourth doses of an NC described herein to a rabbit at 75 umol Mn/kg body weight.

FIG. 9 shows ICP-AES analysis of manganese following first and fourth doses of an NC described herein to a rabbit at 125 umol Mn/kg.

FIG. 10 shows MRI results of an orthotopic breast tumor MDA-MB-231 xenograft mouse model following the IV administration of an NC described herein at 50 μmol Mn/kg. The circle in the bottom right indicates the tumor area.

FIG. 11 shows MRI images following the IV administration of an NC described herein, to detect early stage of brain metastasis of human breast cancer MDA-MB-231 xenografts in a mouse model. Circle and arrow highlight brain metastases.

FIG. 12 compares post-injection T1 weighted MRI signal in a mouse metastatic breast cancer model as between an NC described herein and a commercially available Gd contrast agent.

FIG. 13 shows MRI images following the IV administration of an NC described herein (dose: 35 umol Mn/kg) to detect early stage of brain tumor in a rat U87 glioma model when compared to a commercially available Gd contrast agent (dose: 35 umol Gd/kg) and their respective baseline images. Red circles highlight the brain tumor metastases.

FIG. 14 shows MRI images following the IV administration of an NC described herein (dose: 35 umole Mn/kg) to detect and accurately define tumor rim in an orthotopic rat model of human prostate cancer cell PC3. Circle highlights tumor region.

FIG. 15 shows MRI images following the IV administration to an NC described herein to detect patient-derived primary xenograft pancreatic tumors as compared to a commercially available Gd contrast agent.

FIG. 16 shows (a) the structure of a preferred TERP in the formulation (black; R=polysorbate 80) as complexed with a PSMA targeting agent (blue); (b) ¹H-NMR spectra of Glu-urea-Lys, the uncomplexed TERP and the complexed ter-Glu-urea-Lys (peak overlap is shown by dotted lines); (c) a cartoon depicting how an NC described herein as complexed with a PSMA targeting agent could be expected to target PSMA-positive prostate cancer cells.

FIG. 17 shows confocal microscopy illustrative of cell uptake into PC3 prostate cancer cells with a fluorescently labeled, PSMA targeting NC as compared to a fluorescently labeled, non-PSMA targeting NC.

FIG. 18 shows confocal microscopy illustrative of cell uptake into DU145 prostate cancer cells with a fluorescently labeled, PSMA targeting NC as compared to a fluorescently labeled, non-PSMA targeting NC.

FIG. 19 shows effect of an NC described herein enhance RT efficacy upon a brain tumor.

### DETAILED DESCRIPTION

### Definitions

Unless otherwise defined, terms as used in the specification refer to the following definitions, as detailed below.

The terms “administration” or “administering” compound should be understood to mean providing an NC described herein to an individual in a form that can be introduced into that individual's body in an amount effective for prophylaxis, treatment, or diagnosis, as applicable. Such forms may include e.g., oral dosage forms, injectable dosage forms, transdermal dosage forms, inhalation dosage forms, and rectal dosage forms. In preferred embodiments, the administration is injected.

The term “pharmaceutically acceptable salt” as used herein generally refers to salts prepared from pharmaceutically acceptable non-toxic acids or bases including inorganic acids and bases and organic acids and bases. Suitable pharmaceutically acceptable base addition salts include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art. See, e.g., Remington's Pharmaceutical Sciences, 18 th ed. (Mack Publishing, Easton Pa.: 1990) and Remington: The Science and Practice of Pharmacy, 19 th ed. (Mack Publishing, Easton Pa.: 1995). The preparation and use of acid addition salts, carboxylate salts, amino acid addition salts, and zwitterion salts may also be considered pharmaceutically acceptable if they are, within the scope of sound medical judgment, suitable for use in contact with the tissues of humans and lower animals without undue toxicity, irritation, allergic response, and the like, are commensurate with a reasonable benefit/risk ratio, and are effective for their intended use. Such salts may also include various solvates and hydrates.

Unless otherwise indicated, the terms “prevent,” “preventing” and “prevention” contemplate an action that occurs before a patient begins to suffer from the specified disease or disorder, which inhibits or reduces the severity of the disease or disorder or of one or more of its symptoms. The terms encompass prophylaxis.

Unless otherwise indicated, a “prophylactically effective amount” of a composition is an amount sufficient to prevent a disease or condition, or one or more symptoms associated with the disease or condition, or prevent its recurrence. A prophylactically effective amount of a composition is an amount, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

Unless otherwise indicated, a “diagnostically effective amount” of a composition is an amount sufficient to diagnose a disease or condition. In general, administration of a composition for diagnostic purposes does not continue for as long as a therapeutic use of a composition and might be administered only once if such is sufficient to produce the diagnosis.

Unless otherwise indicated, a “therapeutically effective amount” of a composition is an amount sufficient to treat a disease or condition, or one or more symptoms associated with the disease or condition.

The term “subject” is intended to include living organisms in which disease may occur. Examples of subjects include humans, monkeys, cows, sheep, goats, dogs, cats, mice, rats, and transgenic species thereof.

Parenteral dosage forms may be administered to patients by various routes including subcutaneous, intravenous (including bolus injection), intramuscular, intratumoral, intracranial, and intraarterial. Because their administration typically bypasses patients' natural defenses against contaminants, parenteral dosage forms are specifically sterile or capable of being sterilized prior to administration to a patient. Examples of parenteral dosage forms include solutions ready for injection, dry products ready to be dissolved or suspended in a pharmaceutically acceptable vehicle for injection, suspensions ready for injection, and emulsions. Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like, and suitable mixtures thereof), vegetable oils (such as olive oil) and injectable organic esters such as ethyl oleate, or suitable mixtures thereof. Suitable fluidity of the composition may be maintained, for example, by the use of a coating such as lecithin, by the maintenance of the required particle size in the case of dispersions, and by the use of surfactants. These compositions may also contain adjuvants such as preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form may be brought about by the use of agents delaying absorption, for example, aluminum monostearate and gelatin.

Injectable depot forms may be made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations also are prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic, parenterally acceptable diluent or solvent such as a solution in 1,3-butanediol. Among the acceptable vehicles and solvents that may be employed are water, dextrose solution, 5% dextrose solution, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

In some embodiments, the computation of the total daily dose is based upon the molar quantity of Mn in the composition per kilogram of subject body weight, as opposed to computation using the entire weight of the composition. In some embodiments, the dose of Mn in the composition is standardized during batch manufacture by reference to batch results from inductively coupled plasma atomic emission spectroscopy. In preferred embodiments, the composition is administered to a human at approximately 0.25 to approximately 0.75, more preferably approximately 0.5 umol Mn per kg of body weight.

Disclosed herein is a manufacturing method of polymer-lipid based manganese dioxide nanoparticles that produces NCs in multiple liter scale and, once lyophilized, produces NC powder in multiple gram scale. Details of scale-up, purification, lyophilization, and terminal sterilization are provided that are relevant in providing a pharmaceutical grade precursor material suitable for parental formulation. In some embodiments, the NC is functionalized on its exterior with Glu-urea-Lys, a targeting agent for prostate specific membrane antigen (PSMA) relevant to therapeutic or theranostic use in prostate cancer. In some embodiments, other targeting agents are functionalized upon the exterior of an NC for therapeutic or theranostic use in other cancers. In some embodiments, an NC unfunctionalized by a targeting molecule is used therapeutically or theranostically in cancer. In some embodiments, the cancer is glioblastoma multiforme. In some embodiments, the cancer is prostate cancer.

Also disclosed herein is data supporting the biocompatibility of NC formulations in mammals, including in mouse, rat, rabbit, and dog. Nanoparticle safety is confirmed in vitro in breast, prostate and brain cell lines and in red blood cells.

Also disclosed herein is toxicology and pharmacokinetic data, including tissue exposure and clearance utilizing inductively coupled plasma (ICP) and whole-body MRI, which results are supportive of the use of NC formulations.

Also disclosed herein is the use of NC formulations to enhance imaging or targeting of a tumor using MRI. In some embodiments, NC formulations are used to identify tumor margins. In some embodiments, NC formulations are used to enhance contrast in MRI.

Without wishing to be bound by any particular theory, NCs disclosed herein apparently display enhancement of RT and MRI via interaction with tumoral reactive oxygen species (ROS), such as H₂O₂, and with local O₂/Mn²⁺ generation. NCs release O₂ slowly during the blood circulation phase when there is a low concentration of H₂O₂ but show a concentration-dependent increase in reactivity at higher H₂O₂ concentrations in the weakly acidic tumor microenvironment. Additionally, NCs functionalized with a targeting agent targeting PSMA showed heightened efficacy in a PSMA overexpressing prostate tumor model (functionalized NCs showed a much higher accumulation in prostate tumor cell lines when compared to unfunctionalized NCs). Further demonstration of the utility of the NCs and methods described herein is found in the Examples below.

We have observed that manganese dioxide-based NCs selectively accumulate into tumors, where they reacted with H₂O₂ to release O₂ and soluble Mn²⁺ ions. Such ions have the ability to enhance MRI contrast in the local tumor microenvironment with high sensitivity. In some embodiments, Mn²⁺ ions accomplish this contrast enhancement within 30 min of administration to a subject. We have confirmed this capability preclinically in the vicinity of breast, prostate, brain and pancreatic tumors. In some embodiments, NCs disclosed herein are able to magnetically label the tumor for a longer time than observed with commercially available gadolinium (Gd) based MRI contrast agents (single injection in both cases).

In some manufacturing embodiments, MnO₂ NPs are loaded in TERP/lipid matrix using the oil-in-water (o/w) emulsion method, in combination with a high-pressure homogenization. High pressure homogenization is a technique used in the pharmaceutical industry to obtain suspensions and emulsions for human use. We have successfully utilized this technique to design and develop colloidally stable NCs and scaled up the manufacturing process in a manner easily translatable to commercial production.

In some embodiments, lipids are first mixed with MnO₂ to form an emulsion of MnO₂ nanoparticles in lipid, where the emulsion results due to a balance of electrostatic and hydrophilic/hydrophobic interactions. In some embodiments, an emulsion is then coated in TERP. In some embodiments, an emulsion is processed through high pressure homogenization to improve homogeneity of the emulsion. In some embodiments, a processed emulsion is then washed and concentrated using tangential flow filtration.

In some embodiments, to enhance shelf life, the processed, washed, and concentrated MnO₂/lipid/TERP nanoparticle emulsion is lyophilized using a freeze dryer system in the presence of a cryoprotectant. In some embodiments, the cryoprotectant is glucose. In some embodiments, the cryoprotectant is sucrose. In some embodiments, the cryoprotectant can be glucose, sucrose, mannitole, or trehalose. In some embodiments, the cryoprotectant is mannitol. In some embodiments, the cryoprotectant is present at a 20:1 molar ratio (cryoprotectant:lipids), preferably 10:1 and most preferably 5:1 to 1:1 with respect to the lipids emulsion. In some embodiments, the lyophilized NCs are then irradiated at a dose between 15 and 50 KGy. We have determined that there is no difference in zeta potential or size distribution between pre- and post-irradiated NCs. In some embodiments, size distribution can also be controlled using optimization of cycles and processing pressure when using high pressure homogenization. In some embodiments, the high pressure homogenization involves between 2 and 6 cycles. In some embodiments, the processing pressure for homogenization is between about 15 and about 28 KPsi. In some embodiments, size distribution can also be controlled using optimization of sonication power and time of sonication. In some embodiments, amplitude for sonication is between about 60 and about 100 percent. In some embodiments, time of sonication is between about 2 and about 10 minutes.

In some embodiments, the lipid content of a formulation disclosed herein can be modulated using a range of fatty acids. In some embodiments, the lipid content of a formulation disclosed herein can be modulated using a range of phospholipids. Exemplary fatty acids usable in a formulation disclosed herein comprise Oleic acid, Myristic acid, Ethyl arachidate, Hexadecylamine, Dodecylamine, Caprylic acid, Capric acid, Lauric acid, Stearic acid, Arachidic acid, Behenic acid, Lignoceric acid, Cerotic acid, Erucic acid, Palmitoleic acid, and Sapienic acid. Exemplary phospholipids usable in a formulation disclosed herein comprise 1,2-Didecanoyl-sn-glycero-3-phosphocholine, 1,2-Dierucoyl-sn-glycero-3-phosphate, 1,2-Dierucoyl-sn-glycero-3-phosphate, 1,2-Dipalmitoyl-sn-glycero-3-phosphocholine, 1,2-Dipalmitoyl-sn-glycero-3-phosphate, 1,2-Distearoyl-sn-glycero-3-phosphoethanolamine, 1,2-Distearoyl-sn-glycero-3-phosphoethanolamine, sodium salts thereof, and other pharmaceutically acceptable salts thereof.

In some embodiments, MnO₂ is stabilized with polyvinyl alcohol. In some embodiments, MnO₂ is stabilized with poly(allylamine hydrochloride). In some embodiments, MnO₂ is stabilized with charged polymers, molecules, or proteins. In some embodiments, stabilizing proteins are selected from albumin, IgG, IgE, and IgA.

The formulations described herein can be prepared at room temperature or at temperatures from about room temperature to about 60 degrees Celsius. In some embodiments, the formulations contain NCs having diameters ranging from about 5 nm to about 200 nm. In some embodiments, a formulation is preferentially enriched in NCs having diameters from about 5 nm to about 100 nm, more preferably from about 5 nm to about 80 nm, still more preferably about 20 nm to about 50 nm. In some embodiments, the formulation has a measured negative charge of preferably from −10 to −50 mV.

In some embodiments, TERP is functionalized with a targeting agent prior to being incorporated into the NC. In some embodiments, the targeting agent is an antibody targeting HER2, EGFR, VEGFR, PD-1, PD-L1, CD44, CD133, or some other receptor or protein that is highly expressed in or in the vicinity of cancer cells. In some embodiments, the targeting agent is a peptide targeting PSMA. In some embodiments, the targeting agent is a peptide targeting epidermal growth factor receptors (EGFR) including EGFRVII, low-density lipoprotein receptor (LDLR). In some embodiments, the targeting agent is an aptamer. In some embodiments, the targeting agent is a small molecule. In some embodiments, the targeting agent is folate, which targets the folate receptor. In some embodiments, the targeting agent is hyaluronic acid, which targets CD44 and the receptor for hyaluronin-mediated motility.

In some embodiments, therapeutic methods described herein may be performed using NCs described herein. In some embodiments, therapeutic methods described herein use NCs described in U.S. patent application Ser. No. 16/224,176, which is hereby incorporated by reference.

### EXAMPLES

### Example 1

### Formulation of NCs Using MnO₂ and PVA (Formulation #1)

Formulation #1 Formation of precursor MnO₂ was prepared by direct mixing of potassium permanganate with polyvinyl alcohol (PVA) at room temperature, thereby producing a MnO₂ NP coated with PVA. These NCs were found to have diameters ranging from about 20 nm to about 200 nm.

### Example 2

### Formulation of NCs Using MnO₂ and PAH

A formulation was prepared by direct mixing of potassium permanganate with poly(allylamine hydrochloride) (PAH) at 40-45 degrees Celsius, thereby producing a MnO₂ NP coated with PAH.

### Example 3

### Formulation of a Polymer-Lipid Based MnO₂ NC: T-MX (Formulation #2)

Formulation #2 was obtained by making an oil-in-water emulsion of MnO₂, lipids (phospholipids and cholesterol), and TERP. MnO₂ (30 mM aqueous suspension) was mixed with a lipid and cholesterol mixture (20 mM of 1,2-dipalmitoyl-sn-glycero-3-phosphocholine [DPPC] containing 3% cholesterol, in an ethanol solution) to form manganese dioxide NPs coated with lipids/cholesterol in an oil emulsion. MnO₂ is thus coated with lipid and cholesterol due to electrostatic and a balance of hydrophobic/hydrophilic interactions. TERP (4 mg/mL aqueous) is then added to the emulsion resulting in an additional TERP coating upon the MnO₂/lipid NPs, which we believe follows the same principle of electrostatic interactions and hydrophobic/hydrophilic balance of interactions. The emulsion was processed through high pressure homogenization (pressure: 25 KPsi, 4 cycles) or sonication (amplitude 80%, 10 mins) to obtain polymer-lipid coated nano-size MnO₂. Size distribution was controlled using optimization of cycles and processing pressure when using high pressure homogenization and using sonication power and time of sonication when using ultra high sonication.

### Example 4

### Physical Characterization of T-MX

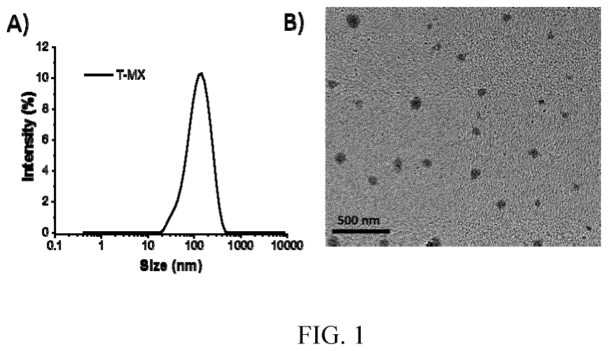

T-MX particle size and zeta potential have been determined using a Malvern ZetaSizer Nano ZS instrument. T-MX in the formulation of Example 3 has particle size 100-140 nm and a narrow size distribution with the PDI at 0.2-0.3 and zeta potential-35±4 (1A). The morphology of T-MX was also confirmed using a Talos L120C transmission electron microscope (TME) (FIG. 1B).

### Example 5

### UV-Vis Spectroscopic Monitoring of T-MX Formation

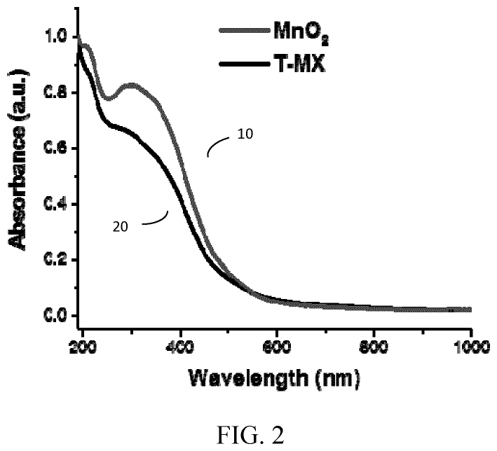

Ultraviolet-visible spectroscopy was used for in-process identification of MnO₂ as compared to formed T-MX. UV-Vis spectrum of MnO₂ and T-MX was obtained using an Agilent UV-Vis system and using water as a reconstitution solvent for both MnO₂ and T-MX. As can be seen in FIG. 2, differences in a broad peak 225 nm-400 nm can be used to identify levels of MnO₂ (line 10) vs T-MX (line 20).

### Example 6

### Thermal Stability of T-MX

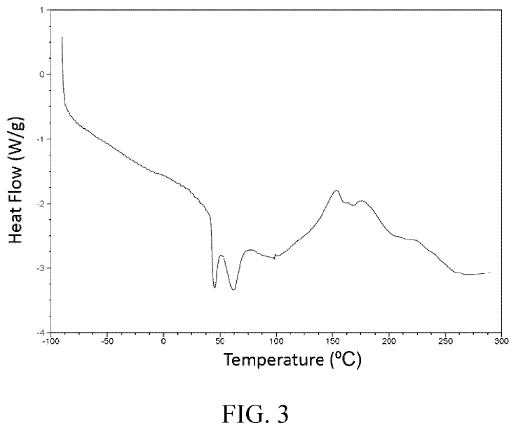

FIG. 3 shows the Differential Scanning calorimetry (DSC) curve of T-MX. The DSC analysis was performed from −90° C.-300° C. at a rate of 30° C./min. The glass transition temperature is above physiologic temperature, demonstrating thermal stability for the formulation.

### Example 7

Reactivity of T-MX Towards H₂O₂

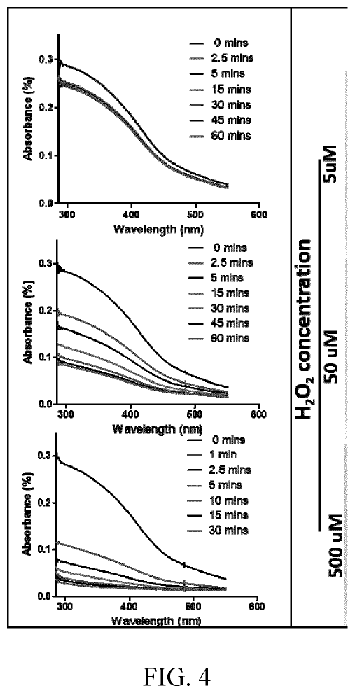

The reactivity of T-MX towards H₂O₂ is confirmed using UV-Vis spectroscopy (FIG. 4). A T-MX formulation at 100 uM showed concentration-dependent reactivity towards H₂O₂. The reactivity profile was observed from 50-500 μM concentration of H₂O₂ to mimic the normal and disease tissue endogenous concentration of H₂O₂. The UV-Vis spectroscopy confirmed the slower reactivity of T-MX at lower concentration of H₂O₂, and the reactivity was significantly increased at higher levels. These data support the in vivo desirability of such NCs in that T-MX displayed the desired programmed high reactivity of T-MX at diseased tissue, but low reactivity during the initial blood circulation time after the IV injection.

### Example 8

### Long-Term Stability of T-MX

Initial stability studies on lyophilized T-MX powder in a Type 1 glass vial showed that the NC when stored at 5±3° C. was stable for up to 6 months. Size, PDI and zeta potential for the lyophilized T-MX powder has shown to be stable. Stability of T-MX is also performed at 5±3° C. and room temperature condition under normal air condition, under vacuum and also sealed in nitrogen gas purged glass vials. The stability in terms of size distribution and zeta potential was also observed unchanged for up to six months.

### Example 9

### In Vitro MRI Characterization of T-MX

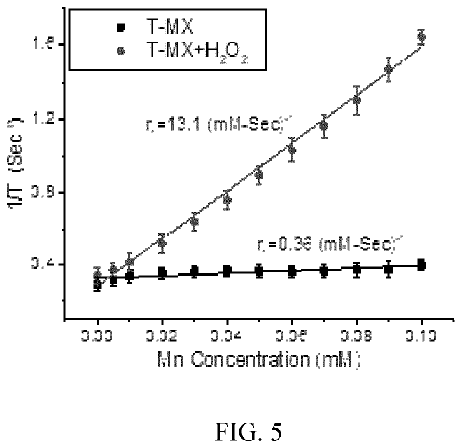

T-MX showed a more than 2.5-fold higher r1 relativity value (13.1 mM⁻¹ sec⁻¹) (FIG. 5) compared to the commercially available gadolinium (Gd) based MR contrast agent, GADOVIST® 1.0 (4.7 mM⁻¹-sec⁻¹). An in vitro MRI of T-MX+H₂O₂ (500 μM) was collected in double de-ionized water at 60 min following H₂O₂ addition at 1.5T. The relaxivity r1 values were obtained from slopes of linear fits of experimental data.

### Example 10

### In Vitro Cytotoxicity of T-MX in Human Breast Epithelial Cells

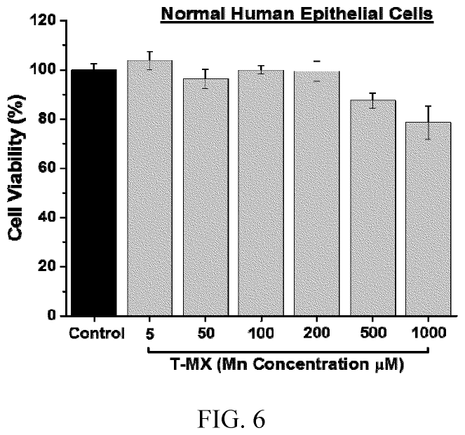

Human breast epithelial cell line (MCF10A) (2×10⁴ cells/well) were exposed to increasing concentrations of T-MX (0, 100, 200, 500 or 1000 μM of Mn) for 24 hr in cell culture media and cell viability was measured using a standard MTT assay (n=5). T-MX was not notably cytotoxic towards human breast cells up to 1000 μM concentration (FIG. 6).

### Example 11

### Hemolysis and Blood Compatibility of T-MX

The hemolytic potential of T-MX will be analyzed using rat, dog, and human whole blood. An initial in vitro hemolysis assay was performed using freshly isolated rat (Sprague Dawley) whole blood processed to a red blood cell (RBC) in PBS, pH 7.4. For the negative and positive controls, RBCs were also treated with equal amounts of PBS or 1% w/v Triton X-100, respectively. At the highest concentration tested T-Mx (2000 μM of Mn) hemolysis was limited to approximately 4%, which indicates no concerning toxicity to red blood cells (approximately 285-fold higher than the estimated Cₘₐₓ (7 μM) for the first-in-human starting dose (0.5 μmole Mn/kg of body weight, corresponds to 35 μmoles of Mn in 70 Kg person with approximately 5 L of blood, that results in maximum Mn blood concentration of 7 μM).

### Example 12

### Uptake and In Vitro Toxicity of T-MX in Cancer Cell Lines

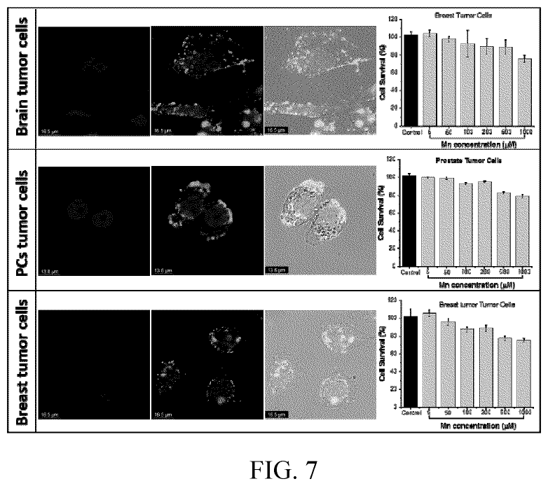

Uptake and in vitro toxicity of T-MX was confirmed in human breast, prostate and brain tumor cell lines. Tumor cells (105 cells, 96 well plate) were incubated for 1-8 hr with fluorescently-labeled T-MX (100 mM) at 37° C. before microscopic analysis. A laser scanning confocal microscope (Zeiss LSM510, Canada) was used to image live cells. Cell nuclei were counter-stained blue with Hoechst 33342 (Invitrogen, Cat #: H3570). FIG. 7 shows intensive accumulation and uniform distribution of T-MX in the cytoplasm and around the nuclei of the cells, depicting internalization and accumulation of T-MX by all breast, prostate and brain tumor cancer cells.

Cancer cell viability was also investigated using a standard methyl thiazoltetrazolium (MTT) assay after 24 hr incubation with increasing concentrations of T-MX at 5-100 μM. The t-MX, does not exert cytototicity on breast, PC3 and brain tumor cell even up to 1000 μM concentration. T-MX NPs were considered nontoxic with cell viability >80% at concentrations of up to 100 μM.

### Example 13

### In Vivo Pharmacokinetics

The pharmacokinetics (PK) of T-MX was evaluated following IV administration to mouse, rat, rabbit and dog. The PK and clearance of T-MX was investigated into healthy mice (BALB/c) and rats (Fischer 344). T-MX showed a distribution half-life at 0.1 hours and an elimination half-life of 8 hours for mouse, as shown in the Tables below:

TABLE 1
Summary of PK parameters

| | Dose | Plasma AUC_{0-∞} | CL | t_{1/2α} | t_{1/2β} | V₁ | Vₛₛ |
|---|---|---|---|---|---|---|---|
| Parameter | (μmol/kg) | (μmol*h/L)^{a, b} | (L/h)^{c} | (h)^{d} | (h)^{d} | (L)^{e} | (L)^{e} |
| Mice | 100 | 615.50 | 4.87 | 0.15 | 7.79 | 3.53 | 47.16 |
| Rat | 37.5 | 77.82 | 0.12 | 0.56 | 10.42 | 0.29 | 1.41 |

^{a}All data points were averaged to provide the mean, and the value was used for calculation; AUC_{0-24 h} was calculated by the trapezoidal rule.
^{b}The blood AUC was obtained from serum samples. AUC_{0-∞} was estimated by addition of AUC_{0-24 h} to AUC_{extrapolated} to infinity or (C_{24 h}/β), where β is the terminal decay constant.
^{c}Total body clearance (CL) was calculated as dose/AUC
^{d}t_{1/2α} represents the distribution half-life and t_{1/2β} represents the elimination half-life.
^{e}Vₛₛ is the volume of distribution at steady state and calculated according to Vₛₛ = V₁ × (1 + k₁₂/k₂₁) where V₁ is the volume of central compartment and k₁₂ and k₂₁ are micro-constants

TABLE 2
Summary of PK parameters

| Parameter | k₁₀ (h⁻¹)^{a,b} | k₁₂ (h⁻¹)^{a,c} | k₂₁ (h⁻¹)^{a,d} | V₁ (L)^{a} | V₂ (L)^{a} |
|---|---|---|---|---|---|
| Mice | 1.96 | 2.67 | 0.21 | 3.53 | 43.60 |
| Rat | 0.47 | 0.64 | 0.17 | 0.29 | 1.11 |

^{a}All data points were averaged to provide the mean, and the value was used for calculation; Data was fitted using two-compartmental model
^{b}k₁₀ is the elimination rate constant.
^{c}k₁₂ is the rate constant from central to peripheral compartment.
^{d}k₂₁ is the rate constant from peripheral to central compartment.

The distribution half-life and elimination half-life of T-MX for rats were 0.5 hr and 10 hr, respectively. Moreover, ICP atomic emission spectroscopy (ICP-AES) of manganese did not exhibit tissue deposition of manganese in major organs at 1 week following the single IV dose of T-MX. T-MX tissue clearance was further confirmed using mouse whole-body MRI (single IV dose, 70 umol/kg). Clearance from bladder and kidney appeared slower than other tissues, potentially indicating a renal route of elimination out to 168 hr.

The plasma clearance profile of T-MX after a single-dose intravenous administration in Beagle dogs was similar to the profiles observed in mice and rats. The blood plasma distribution and elimination half-lives for dog were 0.7 and 6.4 hr, respectively, as determined by ICP-AES quantification of Mn. Further details are given in the Tables below:

TABLE 3
Summary of PK parameters

| | Dose | Plasma AUC_{0-∞} | CL | t_{1/2α} | t_{1/2β} | V₁ | Vₛₛ |
|---|---|---|---|---|---|---|---|
| Parameter | (μmol/kg) | (μmol*h/L)^{a, b} | (L/h)^{c} | (h)^{d} | (h)^{d} | (L)^{e} | (L)^{e} |
| Values | 50 | 44.81 | 8.93 | 0.74 | 6.426 | 12.70 | 25.20 |

^{a}All data points were averaged to provide the mean, and the value was used for calculation; AUC_{0-24 h} was calculated by the trapezoidal rule.
^{b}The blood AUC was obtained from serum samples. AUC_{0-∞} was estimated by addition of AUC_{0-24 h} to AUC_{extrapolated} to infinity or (C_{24 h}/β), where β is the terminal decay constant.
^{c}Total body clearance (CL) was calculated as dose/AUC
^{d}t_{1/2α} represents the distribution half-life and t_{1/2β} represents the elimination half-life.
^{e}Vₛₛ is the volume of distribution at steady state and calculated according to Vₛₛ = V₁ × (1 + k₁₂/k₂₁) where V₁ is the volume of central compartment and k₁₂ and k₂₁ are micro-constants

TABLE 4
Summary of PK parameters

| Parameter | k₁₀ (h⁻¹)^{a,b} | k₁₂ (h⁻¹)^{a,c} | k₂₁ (h⁻¹)^{a,d} | V₁ (L)^{a} | V₂ (L)^{a} |
|---|---|---|---|---|---|
| Values | 0.78 | 0.12 | 0.12 | 12.70 | 12.50 |

^{a}All data points were averaged to provide the mean, and the value was used for calculation; Data was fitted using two-compartmental model
^{b}k₁₀ is the elimination rate constant.
^{c}k₁₂ is the rate constant from central to peripheral compartment.
^{d}k₂₁ is the rate constant from peripheral to central compartment.

### Example 14

### Single-Dose Toxicology of T-MX

The acute toxicity of T-MX was investigated into healthy CD-1 mice, rats (Fischer 344), and Beagle dog by reconstituting T-MX powder in 5CD-1% dextrose solution (sterile, for intravenous use) to an appropriate concentration for dosing.

A non-GLP single-dose IV study in healthy mice (CD-1) was conducted using T-MX at doses of 100, 200, or 300 μmol Mn/kg of body weight. Mortality, clinical chemistry, gross pathology, and histopathology were evaluated. No drug-related mortality was observed in the 14 days recovery period. Clinical chemistry and hematology analysis of serum collected a day 14 post treatment did not show any adverse effects or abnormalities that would indicate drug-related functional impacts on the liver, heart, or kidney up to 300 μmol Mn/kg. See the following Tables for further details:

TABLE 5
Mouse Clinical Chemistry Following Single-Dose IV T-MX

| | | | Experimental Groups | | | | |
|---|---|---|---|---|---|---|---|
| | | | | T-MX (Mn), | | | Reported |
| Panel | Analyte | Unit | Control | 100 μmol/kg | 200 μmol/kg | 300 μmol/kg | Normal Range |
| Liver | ALT | IU/L | 33.3 ± 0.4 | 32.5 ± 8.2 | 39.3 ± 4.9 | 26.2 ± 4.1 | 27-195 |
| Panel | AST | IU/L | 54.8 ± 4.8 | 53.0 ± 6.2 | 79.5 ± 31.4 | 57.9 ± 1.0 | 43-397 |
| | ALP | IU/L | 53.5 ± 1.1 | 46.8 ± 4.4 | 53.9 ± 0.7 | 93.9 ± 12.8 | 35-96 |
| | TBIL | mg/dL | 0.19 ± 0.005 | 0.17 ± 0.01 | 0.17 ± 0.02 | 0.14 ± 0.01 | 0.2-0.6 |
| Cardio- | GLU | g/dL | 377.3 ± 1.5 | 238.5 ± 41.4 | 221.1 ± 13.2 | 205.6 ± 36.8 | 172-372 |
| vascular | CHOL | mg/dL | 194.6 ± 6.1 | 189.4 ± 22.2 | 202.4 ± 28.8 | 164.9 ± 19.8 | 55-169 |
| Panel | HDL | mg/dL | 111.2 ± 0.6 | 108.1 ± 4.9 | 98.8 ± 16.7 | 81.7 ± 4.9 | Not reported |
| | LDL | mg/dL | 3.68 ± 0.33 | 3.89 ± 2.44 | 6.28 ± 0.91 | 5.43 ± 2.23 | Not reported |
| | TRIG | mg/dL | 299.3 ± 12.0 | 238.8 ± 15.4 | 225.4 ± 63.4 | 123.5 ± 12.4 | 67-289 |
| Kidney | TP | g/dL | 6.10 ± 0.15 | 5.71 ± 0.02 | 6.10 ± 0.23 | 5.98 ± 0.26 | 4.8-7.0 |
| Panel | ALB | g/dL | 3.36 ± 0.05 | 3.03 ± 0.07 | 3.60 ± 0.20 | 3.30 ± 0.11 | 2.4-4.3 |
| | PHOS | mg/dL | 8.02 ± 0.02 | 7.63 ± 0.22 | 6.71 ± 0.36 | 6.86 ± 0.90 | 7.3-14.5 |
| | CAL | mg/dL | 10.55 ± 0.27 | 10.27 ± 0.18 | 10.20 ± 0.14 | 10.40 ± 0.14 | 9.7-12.5 |
| | CRE | mg/dL | 0.239 ± 0.012 | 0.200 ± 0.032 | 0.200 ± 0.025 | 0.192 ± 0.020 | 0.2-0.5 |
| | BUN | mg/dL | 29.0 ± 0.4 | 28.2 ± 1.2 | 24.5 ± 2.6 | 26.4 ± 2.9 | 5-28 |
| | Na | mmol/L | 148.6 ± 0.2 | 147.0 ± 2.0 | 148.7 ± 1.1 | 143.6 ± 3.2 | 145.2-181.2 |
| | K | mmol/L | 5.58 ± 0.11 | 5.44 ± 0.27 | 5.24 ± 0.56 | 4.97 ± 0.44 | 7.27-11.18 |
| | Cl | mmol/L | 103.3 ± 0.5 | 102.3 ± 2.1 | 103.8 ± 1.7 | 104.9 ± 2.5 | 110.7-134.0 |

TABLE 6
Mouse Hematology Following Single-Dose IV T-MX

| | | Experimental Groups | | | | |
|---|---|---|---|---|---|---|
| | | | T-MX (Mn) | | | Reported |
| Analyte | Unit | Control | 100 μmol/kg | 200 μmol/kg | 300 μmol/kg | Normal Range |
| WBC | 10³/uL | 4.95 = 0.01 | 4.71 = 2.48 | 3.58 = 1.21 | 5.27 = 2.07 | 3.90-13.96 |
| RBC | 10⁶/uL | 7.78 = 1.08 | 7.14 = 1.60 | 7.53 = 1.37 | 8.76 = 0.35 | 7.14-12.20 |
| Hgb | g/dL | 11.25 = 1.55 | 9.90 = 2.43 | 10.80 = 2.05 | 12.40 = 0.37 | 10.8-19.2 |
| HCT | % | 41.50 = 4.40 | 37.77 = 8.23 | 39.13 = 7.92 | 44.37 = 1.72 | 37.2-62.0 |
| MCV | fL | 53.55 = 1.75 | 53.00 = 2.38 | 51.77 = 2.71 | 50.77 = 3.77 | 42.6-56.0 |
| MCH | pg | 14.45 = 0.05 | 13.80 = 0.37 | 14.30 = 0.99 | 14.13 = 0.12 | 11.7-16.8 |
| MCHC | g/dL | 27.00 = 0.90 | 26.17 = 1.74 | 27.67 = 0.76 | 27.97 = 1.75 | 24.6-35.9 |
| PLT | 10³/uL | 698.00 = 141.00 | 565.50 = 170.50 | 428.50 = 86.50 | 676.00 = 64.75 | 555-2159 |
| MPV | fL | 5.00 = 0.10 | 4.83 = 0.26 | 5.30 = 0.71 | 4.83 = 0.47 | 4.3-6.1 |
| RDW | % | 16.70 = 0.10 | 17.13 = 0.37 | 16.47 = 0.63 | 17.1 = 0.67 | 15.9-21.1 |
| NE | % | 25.07 = 8.98 | 18.27 = 4.11 | 17.60 = 0.53 | 27.40 = 1.59 | 7.36-28.59 |
| LY | % | 70.39 = 6.92 | 76.40 = 2.56 | 73.80 = 1.84 | 67.66 = 1.32 | 61.26-87.82 |
| MO | % | 3.80 = 1.87 | 3.66 = 1.02 | 6.41 = 1.43 | 4.60 = 0.51 | 2.18-11.02 |
| EO | % | 0.60 = 0.06 | 1.34 = 0.82 | 1.69 = 1.31 | 0.31 = 0.17 | 0.13-4.51 |
| BA | % | 0.15 = 0.12 | 0.33 = 0.19 | 0.51 = 0.41 | 0.03 = 0.03 | 0.01-1.26 |
| NE | 10³/uL | 1.24 = 0.45 | 0.96 = 0.71 | 0.63 = 0.20 | 1.41 = 0.47 | 0.42-3.09 |
| LY | 10³/uL | 3.49 = 0.33 | 3.54 = 1.74 | 2.66 = 0.95 | 3.60 = 1.48 | 2.88-11.15 |
| MO | 10³/uL | 0.19 = 0.09 | 0.15 = 0.04 | 0.22 = 0.06 | 0.24 = 0.10 | 0.15-0.94 |
| EO | 10³/uL | 0.03 = 0.00 | 0.04 = 0.01 | 0.06 = 0.04 | 0.02 = 0.02 | 0.01-0.50 |
| BA | 10³/uL | 0.01 = 0.01 | 0.01 = 0.005 | 0.02 = 0.02 | 0.003 = 0.003 | 0.00-0.14 |

The histopathology evaluation of major body organs of mice collected at day 14 post dose were viewed and evaluated by a board-certified pathologist. This evaluation did not show any gross finding considered being treatment-related and microscopic histopathology of major body organs confirmed no drug-related adverse effects.

A non-GLP single-dose IV study in healthy rats (Fischer 344) was conducted using T-MX at doses of 120, 235, 300, or 350 μmol Mn/kg. Mortality, gross pathology, and histopathology were evaluated at day 14 post treatment. No drug-related mortality was observed during the recovery period. The clinical biochemistry data indicated no significant biomarker level differences were identified comparing a single dose IV treatment of T-MX up to the dose of 350 μmol Mn/kg b.w., when compared to the control groups. See the following Tables for further details:

TABLE 7
Rat Clinical Chemistry Following Single-Dose IV T-MX

| | | | Experimental Groups | | | |
|---|---|---|---|---|---|---|
| | | | | T-MX (Mn) | | |
| Panel | Analyte | Unit | Control | 120 μmol/kg | 235 μmol/kg | 350 μmol/kg |
| Liver | ALT | IU/L | 54.80 | 81.20 | 66.35 | 38.95 |
| Panel | AST | IU/L | 55.10 | 56.66 | 115.95 | 86.50 |
| | ALP | IU/L | 214.60 | 133.10 | 137.10 | 157.60 |
| | TBIL | mg/dL | 0.12 | 0.31 | 0.13 | 0.16 |
| Cardio- | GLU | mg/dL | 237.10 | 173.50 | 213.25 | 208.80 |
| vascular | CHOL | mg/dL | 91.10 | 101.30 | 115.10 | 110.90 |
| Panel | HDL | mg/dL | 35.10 | 33.80 | 36.40 | 38.30 |
| | LDL | mg/dL | 2.57 | 3.83 | 4.87 | 4.87 |
| | TRIG | mg/dL | 146.50 | 117.45 | 79.90 | 56.55 |
| Kidney | TP | g/L | 63.20 | 66.25 | 70.00 | 64.00 |
| Panel | ALB | g/L | 36.80 | 39.15 | 34.01 | 38.55 |
| | PHOS | mg/dL | 7.74 | 9.13 | 8.85 | 7.62 |
| | CAL | mg/dL | 3.67 | 0.83 | 2.01 | 3.65 |
| | CRE | mg/dL | 0.38 | 0.41 | 0.50 | 0.36 |
| | BUN | mg/dL | 24.10 | 22.50 | 24.70 | 19.00 |
| | Na | mmol/L | 139.10 | 133.10 | 139.50 | 142.30 |
| | K | mmol/L | 12.06 | 28.71 | 17.76 | 12.12 |
| | Cl | mmol/L | 101.30 | 98.65 | 100.15 | 102.00 |

The histopathology evaluation of major body organs of mice collected at day 14 post dose were viewed and evaluated by a board-certified pathologist. This evaluation did not show any gross pathologic findings of the major organs and no adverse histopathology observations up to the dose of 350 μmol Mn/kg body weight.

A non-GLP single-dose toxicity study was performed in Beagle dogs in order to assess the maximum tolerated dose for T-MX. Dogs were administered single-dose IV infusion (rate of 3 mL/min) of T-MX (Lot No. 0819) at 105 or 110 μmole Mn/kg for males (2), or 99 μmole Mn/kg females (1), respectively. Mortality, clinical observation (once daily), body weight (Days 1, 7, 14), food consumption (daily), hematology, and clinical chemistry were evaluated prior to euthanasia (Day 15-16), as well as gross and histopathology of selected organs (brain, heart, liver, lungs, spleen, kidneys, intestines, bladder, and skeletal muscle). Following dosing, whole blood and plasma were collected at predose, and at 0.5, 1, 4, 8, and 24 hr post dose for possible Mn assessment using ICP-AES. End of study tissue samples were also collected and snap-frozen for possible Mn assessment, and the remaining tissues were evaluated for gross pathology and prepared for histopathological assessment. No mortality was observed during this study, and there were no adverse effects on body weight or food consumption over the 14-day observation period. Clinical signs following IV administration to dogs including struggling during dosing, mild salivation, involuntary urination, mild tachypnoea, involuntary bowel movements, sedation (ranging from mild to severe), labored breathing, and mild vomiting. All animals appeared normal by 1 hour 45 minutes to 4 hours post dose. At the end of the observation period on Days 15-16, some clinical chemistry parameters decreased below the normal range including Ca, Phos, and CK; however, these excursions were very minor and were not considered to be clinically significant. Some hematology parameters remained outside of the normal range including % EOS in males and RBC, HEMO, HEMA, BAS, and BAS % in females; however, these excursions were very minor. The hematological assessment and blood clinical chemistry assessment indicate no adverse drug-related effects on parameters related liver, kidneys, and cardiac functions at 15-16 days after the IV administration of T-MX. The maximum tolerated dose (MTD) for T-MX by IV infusion at a rate of 3 mL/min for Beagle dogs was considered 110 μmole Mn/kg (6.2 mg/kg Mn) for males and 99 μmole Mn/kg (5.5 mg/kg Mn) for females. See the following Tables for further details:

TABLE 13
Dog Hematology Following Single-Dose IV T-MX

| | | | | T-MX (Mn) | |
|---|---|---|---|---|---|
| | | Normal range | | Male | Female |
| Analyte | Unit | Male | Female | 110 μmol/kg | 99 μmol/kg |
| RBC | 10¹²/L | 5.8-7.44 | 6.13-7.79 | 7.21 | 8.16 |
| HEMO | g/L | 133-168 | 137-174 | 164 | 181 |
| HEMA | (%) | 40-51.5 | 40.9-53.5 | 51.2 | 54.6 |
| MCV | fL | 63.5-71.4 | 62.6-72.7 | 71 | 66.9 |
| MCH | pg | 21.3-23.8 | 20.8-23.9 | 22.8 | 22.2 |
| MCHC | g/L | 318-342 | 315-344 | 321 | 332 |
| PLAT | 10⁹/L | 233-440 | 216-486 | 175 | 203 |
| MPV | fg | — | — | 9.5 | 9.9 |
| WBC | 10⁹/L | 6.87-14.04 | 7.21-14.53 | 12.57 | 8.77 |
| NEU | 10⁹/L | 3.61-8.76 | 3.16-9.11 | 6.52 | 5.83 |
| NEUT | % | 45.2-66.2 | 43.7-66.7 | 51.9 | 66.5 |
| LYM | % | 24.1-43.7 | 23.7-46 | 38.5 | 23.5 |
| MO | % | 4.4-8.7 | 3.6-8 | 4.7 | 4.4 |
| EOS | % | 0.8-5.5 | 0.6-4.6 | 4.4 | 1.1 |
| BAS | % | 0.4-3.0 | 0.4-3.2 | 2.3 | 4.1 |
| LUC | % | 0.2-0.7 | 0.2-0.8 | 0.6 | 0.5 |
| RET | % | 0.24-1.07 | 0.24-0.98 | 0.61 | 0.57 |

TABLE 14
Dog Clinical Chemistry Following Single-Dose IV T-MX

| | | | Experimental Groups | | |
|---|---|---|---|---|---|
| | | | | T-MX (Mn) | |
| | | Normal range | | Male | Female |
| Analyte | Unit | Male | Female | 110 μmol/kg | 99 μmol/kg |
| A/G | | 1.1-1.5 | 1.1-1.7 | 1.1 | 1.2 |
| ALB | g/L | 27-35 | 27-37 | 31 | 32 |
| GLOB | g/L | 22-29 | 19-27 | 27 | 26 |
| ALKP | U/L | 50-149 | 52-156 | 113 | 86 |
| TBIL | μmol/L | 1.7-4.1 | 1.7-4.6 | 1.7 | 2.5 |
| BUN | mmol/L | 3.2-5.9 | 3.6-7.1 | 4.7 | 6.2 |
| Ca | mmol/L | 2.65-3.67 | 2.68-3.76 | 2.62 | 2.65 |
| Cl | mmol/L | 109-119 | 109-122 | 112 | 113 |
| CREA | μmol/L | 45-67 | 42-66 | 58 | 62 |
| GLU | mmol/L | 4.2-6.4 | 4.3-6.2 | 5.5 | 5.2 |
| LDH | U/L | 124-617 | 125-689 | 100 | 137 |
| Phos | mmol/L | 1.69-2.4 | 1.65-2.44 | 1.3 | 1.46 |
| K | mmol/L | 4.2-5.4 | 4.3-5.5 | 4.8 | 4.9 |
| TP | g/L | 50-63 | 50-61 | 58 | 59 |
| AST | (U/L) | 25-46 | 29-55 | 25 | 36 |
| ALT | U/L | 33-61 | 29-59 | 81 | 57 |
| Na | mmol/L | 141 157 | 141 161 | 142 | 144 |
| TRIG | mmol/L | 0.19-0.48 | 0.17-0.57 | 0.58 | 0.46 |
| CK | U/L | 95-291 | 92-437 | 47 | 100 |
| CHOL | mmol/L | 2.77-4.73 | 2.61-4.26 | 3.36 | 3.18 |
| CGT | U/L | 5.0-10.0 | 5.0-9.0 | 15 | 8 |

### Example 15

### Repeat-Dose Toxicology of T-MX

A non-GLP repeat-dose toxicity (4×1 per week) study was also performed in healthy rats (Sprague Dawley). The study was conducted using T-MX at dose 300 μmol Mn/kg. For this study animals were injected IV once every weeks for four weeks. Animals were recovered for 14 days post last treatment. In this study mortality, gross pathology, and histopathology were evaluated at day 14 post last treatment of T-MX. No drug-related mortality was observed during the recovery period. The clinical biochemistry and hematology of serum were performed at 24 hours post last treatment of T-MX. The serum hematology and biochemistry data indicated no significant biomarker level differences and values were observed when comparing the repeat dose IV treatment of T-MX at the dose of 300 μmol Mn/kg with the control groups. See the following Tables for further details:

TABLE 8
Rat Serum Hematology Following Repeat-
Dose IV Treatment of T-MX

Experimental Groups

| | |
|---|---|
| | T-MX (Mn) |
| Control (Terpolymer) | (300 μmol/kg) |

| Analyte | Unit | Female | Male | Female | Male |
|---|---|---|---|---|---|
| WBC | 10³/L | 11.70 | 10.56 | 4.94 | 15.86 |
| RBC | 10⁶/L | 8.87 | 8.20 | 7.14 | 9.16 |
| Hgb | g/dL | 16.50 | 16.30 | 13.50 | 16.00 |
| HCT | % | 55.40 | 48.30 | 42.50 | 52.67 |
| MCV | fL | 62.50 | 58.90 | 59.50 | 57.50 |
| MCH | pg | 18.60 | 19.90 | 18.90 | 17.50 |
| MCHC | g/dL | 29.80 | 33.70 | 31.80 | 30.43 |
| MPV | fL | 6.50 | 7.90 | 8.00 | 6.07 |
| RDW | % | 14.60 | 15.20 | 15.70 | 14.73 |
| NE | % | 40.39 | 14.71 | 31.80 | 30.43 |
| LY | % | 50.44 | 78.97 | 55.12 | 48.50 |
| MO | % | 8.18 | 5.45 | 10.35 | 2.65 |
| EO | % | 0.87 | 0.82 | 1.38 | 1.08 |
| BA | % | 0.13 | 0.05 | 0.45 | 0.13 |

TABLE 9
Rat Clinical Chemistry Following
Repeat-Dose IV treatment of T-MX

| | | | Experimental Groups | | | |
|---|---|---|---|---|---|---|
| | | | Control | | T-MX (Mn) | |
| | | | (Terpolymer) | | (300 μmol/kg) | |
| Panel | Analyte | Unit | Female | Male | Female | Male |
| Liver | ALT | IU/L | 71.80 | 95.60 | 66.20 | 59.10 |
| Panel | AST | IU/L | 99.50 | 142.70 | 141.40 | 80.80 |
| | ALP | IU/L | 123.90 | 118.60 | 122.20 | 49.60 |
| | TBIL | mg/dL | 1.10 | 1.10 | 2.00 | 1.00 |
| Cardio- | GLU | mg/dL | 615.30 | 144.70 | 620.30 | 318.20 |
| vascular | CHOL | mg/dL | 110.80 | 114.90 | 125.10 | 95.47 |
| Panel | HDL | mg/dL | 37.40 | 28.80 | 44.90 | 58.01 |
| | LDL | mg/dL | 1.52 | 1.08 | 2.89 | 4.46 |
| | TRIG | mg/dL | 66.90 | 50.90 | 60.00 | 55.60 |
| Kidney | TP | g/dL | 7.58 | 6.99 | 6.72 | 3.43 |
| Panel | ALB | g/dL | 3.00 | 3.88 | 3.00 | 1.54 |
| | PHOS | mg/dL | 12.30 | 10.40 | 18.50 | 4.78 |
| | CAL | mg/dL | 13.60 | 11.00 | 10.20 | 3.60 |
| | CRE | mg/dL | 0.55 | 1.90 | 0.96 | 0.25 |
| | BLN | mg/dL | 19.70 | 25.30 | 16.70 | 15.30 |
| | Na | mmol/L | 138.30 | 133.70 | 140.00 | 100.70 |
| | K | mmol/L | 12.96 | 9.57 | 6.31 | 6.08 |
| | Cl | mmol/L | 91.90 | 95.00 | 99.80 | 74.20 |

The histopathology evaluation of major body organs of mice collected at day 14 post dose were viewed and evaluated by a board-certified pathologist. This evaluation did not show any gross pathologic findings of the major organs and no adverse histopathology observations up to the dose of 300 μmol Mn/kg body weight.

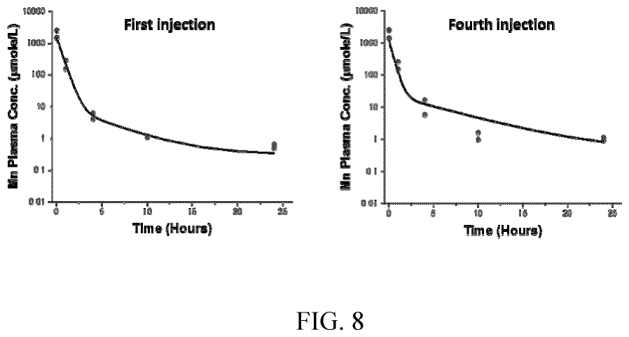

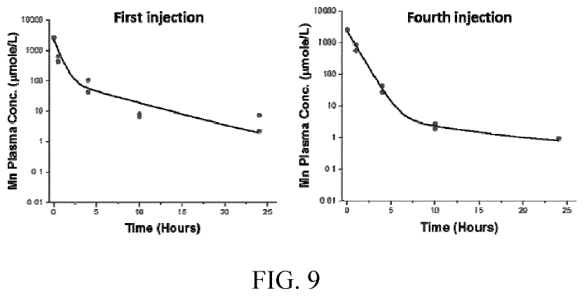

A non-GLP four-week, once weekly intravenous toxicity and toxicokinetics study of T-MX was conducted in healthy rabbits (female New Zealand white rabbits) by a third-party contract research organization (CRO), Nucro-Technics (Ontario, Canada). The study was conducted using T-MX at mid dose (75 μmol Mn/kg), high dose (125 μmol Mn/kg), and TERP at a dose ˜60 mg/kg (novel excipient control). For this study animals were injected IV once every week for four weeks. Clinical pathology evaluation (hematology and clinical chemistry) was performed on Days 5 and 24 (dosing started on Day 1) and serial blood sampling for determination of Mn in plasma occurred on Days 1 and 22 (for the first and last of four doses) at: prior to dosing; immediately after dose; and at 1, 4, 10, and 24 hours post dosing. Lastly, necropsy was performed on Day 29 and select tissues were preserved for Mn bioanalysis and histopathology. No significant change in Mn concentration was observed, indicating a lack of Mn accumulation in the major tissues (brain, lungs, liver, bladder, heart, spleen, kidneys and jejunum) following repeated doses of the test item at two different dose levels when compared to control rabbits. Clearance of Mn from the body appears to be relatively quick and is supported by toxicokinetic (TK) data; see FIGS. 8 and 9. Following the 1st dose (Day 1) and 4th dose (Day 22) of T-MX as fitted to a two-compartment model, the distribution half-life of the Mn (t1/2α) was approximately 0.3 hr to 0.6 hr, while the elimination half-life was approximately 3 hr to 6 hr for both mid and high doses (t1/2β). No significant changes in distribution half-life and elimination half-life were observed following multiple dose administrations of T-MX. Importantly, there were no toxicological findings relevant to the exposure of T-MX on clinical pathology, necropsy, or histopathology and no severe toxicity was recognized related to the vehicle (TERP) and T-MX at high doses. Key hematology and serum clinical biochemistry parameters were measured from blood collected at 48 hours post last (4th) IV treatment. Both hematology and clinical biochemistry data indicated that all tested biomarkers were within the reported normal range, with no significant biomarker level differences were identified following the repeat dose IV treatment of T-MX at 75 and 125 μmol Mn/kg b.w. Please see the following Tables for further details:

TABLE 10
Summary of TK parameters

| | Mid dose | Mid dose | High dose | High dose |
|---|---|---|---|---|
| | 1^{st} | 4^{th} | 1^{st} | 4^{th} |
| Parameters | Injection | Injection | Injection | Injection |
| Dose (μmol/kg) | 75 | 75 | 125 | 125 |
| AUC_{0-∞} (μmol*h/L) | 1239 | 1111 | 2818 | 2892 |
| C_{EOI} (μmol/L) | 1505 | 1440 | 2619 | 2560 |
| CL (L/h) | 0.242 | 0.269 | 0.177 | 0.172 |
| t_{1/2α} (h) | 0.356 | 0.290 | 0.422 | 0.635 |
| t_{1/2β} (h) | 2.828 | 4.001 | 3.949 | 5.986 |
| V₁ (L) | 0.197 | 0.219 | 0.215 | 0.210 |
| V₂ (L) | 0.063 | 0.442 | 0.348 | 0.035 |
| k₁₀ (h⁻¹) | 1.847 | 1.910 | 1.179 | 1.069 |
| k₁₂ (h⁻¹) | 0.083 | 0.435 | 0.395 | 0.019 |
| k₂₁ (h⁻¹) | 0.257 | 0.216 | 0.245 | 0.118 |
| Vₛₛ (L) | 0.261 | 0.661 | 0.563 | 0.245 |

Note:
values within a group were averaged to provide a mean result.
AUC_{0-∞}: area under the concentration curve from time zero extrapolated to infinity
C_{EOI}: plasma concentration at the end of infusion (immediately after dosing)
CL: total body clearance
k10: elimination rate constant
k12: rate constant from central to peripheral compartment
k21: rate constant from peripheral to central compartment
t1/2α: distribution half-life
t1/2β: elimination half-life
V1: volume of central compartment
V2: volume of peripheral compartment
Vss: volume of distribution at steady state

TABLE 11
Rabbit Serum Hematology Following Repeat-Dose IV Treatment of T-MX

| | | | Experimental Groups | | |
|---|---|---|---|---|---|
| | | | Terpolymer | T-MX (Mn) | |
| Analyte | Unit | Normal range | Control | 75 μmol/kg | 125 μmol/kg |
| RBC | 10¹²/L | 5.42-7.02 | 5.77 | 5.76 | 5.66 |
| HEMO | g/L | 113-143 | 132 | 126 | 120 |
| HEMA | (%) | 34.2-43.2 | 38.2 | 38.7 | 35.4 |
| MCV | fL | 57.4-67.1 | 66.3 | 67.2 | 62.2 |
| MCH | pg | 18.9-22.4 | 22.8 | 21.7 | 21.2 |
| MCHC | g/L | 312-352 | 344 | 324 | 338 |
| PLAT | 10⁹/L | 179-610 | 294 | 404 | 375 |
| WBC | 10⁹/L | 3.53-10 | 6.87 | 8.16 | 8.96 |
| NEU | 10⁹/L | 0-2.52 | 1.03 | 1.75 | 1.74 |
| NEUT | % | — | 14.8 | 21.5 | 19.3 |
| LYM | 10⁹/L | 2.41-7.16 | 4.66 | 5.32 | 5.82 |
| MO | 10⁹/L | 0-0.35 | 0.39 | 0.45 | 0.66 |
| EOS | 10⁹/L | 0.04-0.24 | 0.12 | 0.08 | 0.09 |
| BAS | 10⁹/L | 0.040-0.72 | 0.57 | 0.46 | 0.55 |
| LUC | 10⁹/L | 0-0.09 | 0.1 | 0.1 | 0.1 |
| RET | 10⁹/L | 49.7-236.4 | 167.1 | 247 | 233 |

TABLE 12
Rabbit Serum Clinical Chemistry Following Repeat-Dose IV Treatment of T-MX

| | | | Experimental Groups | | |
|---|---|---|---|---|---|
| | | | Terpolymer | T-MX (Mn) | |
| Analyte | Unit | Normal range | Control | 75 μmol/kg | 125 μmol/kg |
| A/G | | 1.6-3 | 1.5 | 1.4 | 1.6 |
| ALB | g/L | 28-42 | 30 | 30 | 32 |
| GLOB | g/L | 11-20 | 20 | 21 | 20 |
| ALKP | U/L | 38-202 | 121 | 20 | 62 |
| TBIL | μmol/L | 1.7-6.7 | 4.2 | 4.5 | 2.6 |
| BUN | mmol/L | 2.2-8.4 | 6.8 | 7.6 | 7.6 |
| Ca | mmol/L | 2.83-3.71 | 3.6 | 3.65 | 3.7 |
| Cl | mmol/L | 97-111 | 110 | 110 | 110 |
| CREA | μmol/L | 40-117 | 66 | 66 | 66 |
| GLU | mmol/L | 4.7-8.4 | 7.6 | 7.4 | 7.6 |
| Phos | mmol/L | 1.41-3.05 | 1.38 | 1.21 | 1.52 |
| K | mmol/L | 3.5-5.7 | 4.8 | 4.9 | 5.1 |
| TP | g/L | 42-60 | 56 | 60 | 58 |
| AST | (U/L) | 6-43 | 14 | 18 | 13 |
| ALT | U/L | 25-73 | 37 | 48 | 60 |
| Na | mmol/L | 133-148 | 142 | 143 | 144 |
| TRIG | mmol/L | 0.29-1.44 | 1.16 | 0.99 | 1.16 |
| CHOL | mmol/L | 0.84-1.77 | 2.36 | 1.41 | 1.72 |

### Example 16

### MRI Contrast Enhancement and Tumor Imaging Using T-MX

All in vivo magnetic resonance imaging (MRI) acquisition utilized a 7 Tesla preclinical MRI system (70/30 USR Biospec, Bruker Corporation), implemented with the B-GA12 gradient coil insert. Each rodent was positioned prone on a dedicated mouse or rat scanning bed including inlaid water tubes for temperature regulation and isoflurane (1.8% in oxygen) delivered to a nose cone. A pneumatic pillow provided a respiratory trace for monitoring and gated acquisitions (SA Instruments, Stony Brook, NY). Contrast agent was delivered via tail vein using an automated high precision MR-compatible syringe pump (Harvard Apparatus, Holliston, MA). Animals are also prepared via tail vein cannulation with a 27 G needle and a precision line (80 μL internal volume), to enable manual contrast injection following baseline scanning. Except for the whole body protocol (T1-weighted imaging only), a 2D T2-weighted image set guided the prescription of a T1-weighted image set with matching geometric feature. The T1-weighted images were used for visualizing contrast agent distribution and clearance.

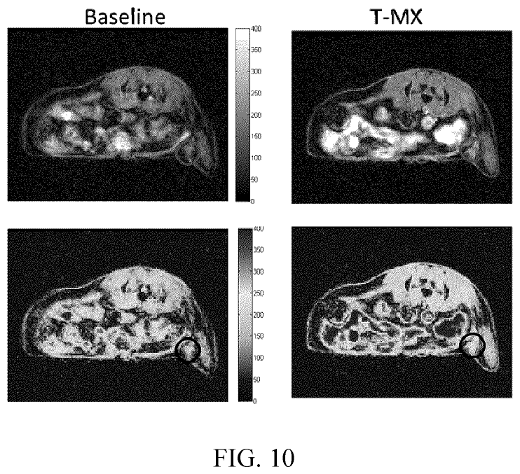

FIG. 10 shows the MRI results of an orthotopic breast tumor MDA-MB-231 xenograft mouse model following the IV administration of T-MX (50 μmole Mn/kg bw). MRI images were acquired at 30 mins post-administration of T-MX. T-MX was able to detect the early stage breast tumor after 7 days following inoculation of tumor cells in the mouse mammary fat pad.

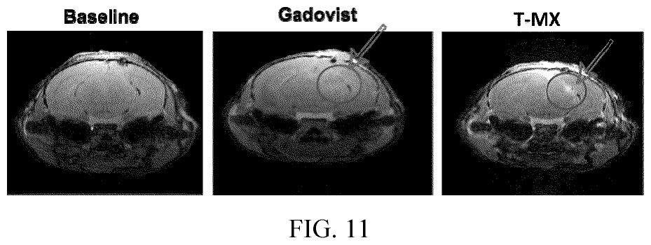

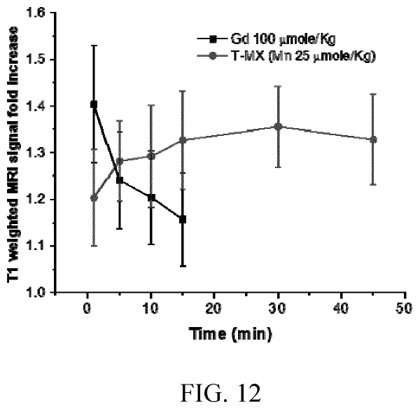

To explore potential application of T-MX as an MRI contrast agent, T-MX was administrated IV into different models of human cancer cells-derived tumors. FIG. 11 shows the pre-clinical mouse model of brain metastasis of human breast cancer MDA-MB-231 mouse model. The investigation was started after 7 days following inoculation of tumor cells in the mouse brain. The MRI signal was compared to commercially available Gd based contrast agent Godavist (gadobutrol). As seen in FIG. 12, 3 mins post-administration of Gadovist, the brain tumor was not detectable using Gd; however T-MX was able to detect the brain tumor with high accuracy. T-MX provided detection of the early stage of brain tumor metastasis at a dose of 25 umole Mn/kg, 4 times lower than Gadovist (100 umol/kg, approved gadolinium-based MRI contrast agent). Interestingly, the brain MRI signal for T-MX was stable up to 1 hour, while Gd signal began decreasing rapidly between 0 and 5 min post-dose. Signal stability of Manganoscan is desirable for real-time imaging during MRI guided radiotherapy of tumors and these results suggest that multiple administration of T-MX may not be needed.

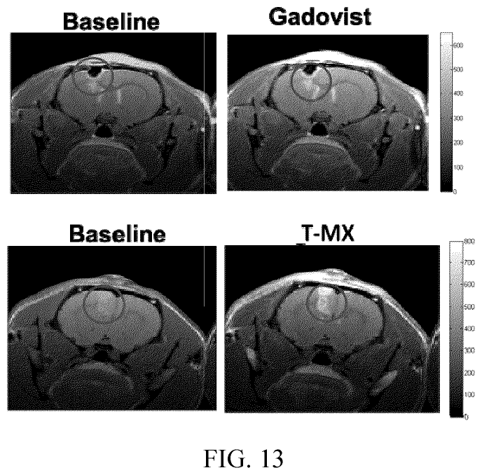

Similarly, a rat human U87 Glioma model was established by inoculation of human glioblastoma clone U87 cells into immunosuppressed rat brain and the MRI study was started after 7 days following the inoculation. T-MX showed much higher detection accuracy at the same dose compared to Gadovist™ (35 umole/kg) in the rat human U87 Glioma model, as shown in FIG. 13.

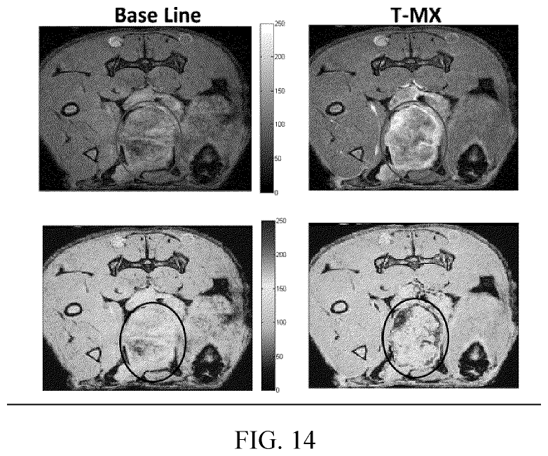

Further, T-MX properties were studied in prostate cancer. FIG. 14 shows the MRI results of an orthotropic rat model of human prostate cancer cell PC3 following injection with T-MX at a dose of 35 umole Mn/kg. As seen in the figure T-MX was able to detect the tumor margin precisely. We believe, due to nano-sized properties of T-MX and the abnormal vasculature system in the tumor area, T-MX has superior accumulation and is retained longer in the tumor area. Such an observation is consistent with referring to the effect as “Enhanced Permission and Retention” (EPR). T-MX is able to precisely delineate the tumor rim within minutes of injection.

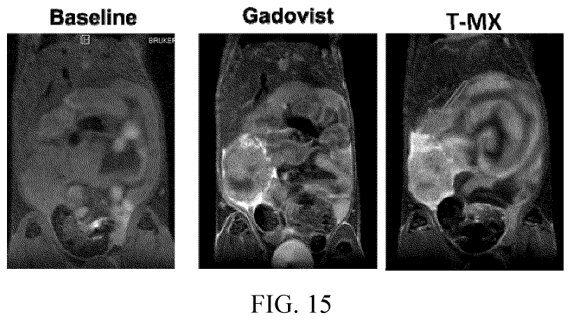

Still further, T-MX properties were studied in pancreatic cancer (FIG. 15). T-MX was also applied to detect the tumor margins using MRI for a pancreatic xenograft tumor and compared to the administration of commercially available Gadovist at the same dose (100 μmol Mn/kg). T-MX was able to detect the tumor area that was not detectable by Gadovist.

### Example 17

### Synthesis and Evaluation of T-MX Formulation Including a PSMA Targeting Agent

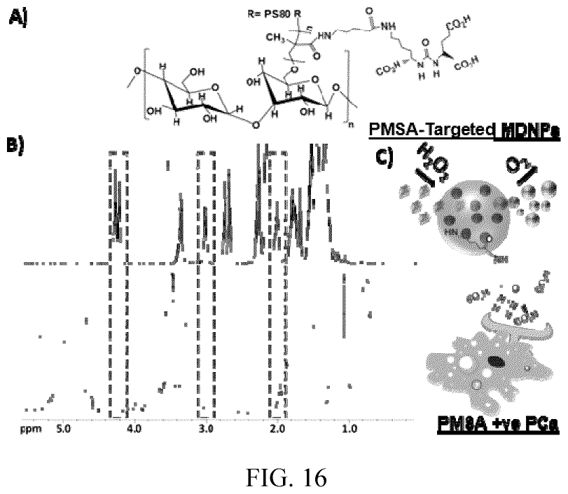

A T-MX formulation including a PSMA targeting agent is obtained by functionalizing TERP with the targeting agent prior to being added to the formulation. For this purpose, Glu-Urea-Lys is obtained and covalently conjugated with TERP. The small molecules have an active amine (NH₂) group that can be covalently conjugated to carboxylic acid group (COOH) without altering the targeting ability of the ligand molecule. As can be seen in FIG. 16A, the preferred TERP has a large number of active COOH groups on its polymethacrylic acid side chains that are useful for such conjugations. At the first step, Glu-urea-Lys molecules is successfully attached with TERP using EDC/NHS chemistry. The Glu-urea-Lys functionalized TERP (ter-Glu-urea-Lys) is purified using dialysis and lyophilized for further characterization and synthesis of PSMA targeting T-MX. Conjugation is validated by 1H-proton NMR, where PSMA peaks were observed in 2.0-5.0 ppm range for final PSMA-TERP product FIG. 16B.

PSMA ligand functionalized TERP (ter-Glu-urea-Lys) was used to synthesize PSMA targeting T-MX. For the NP synthesis the precursor MnO₂ was loaded with TERP-Glu-urea-Lys/Lipid matrix using the oil-in-water (o/w) emulsion method, in combination with high pressure homogenizer technique.

We optimized the synthesis conditions by using different lipids, adjusting the pressure (1000-30000 KPsi), preferably from 10000-30000 KPsi and most preferably from 20000-300000 KPsi and cycles one to eight, preferably from two to six and most preferably from two to four of high pressure homogenizer to obtain stable T-MX suspension of small size, and narrow polydispersity index (PDI) suitable for intravenous (IV) injection. Lyophilized conditions were optimized to obtain T-MX powder that will improve their stability and increase shelf-life. Different cryoprotectants (glucose, sucrose, trehalose and mannitol) were used to obtain dry T-MX without aggregation and altering their physical properties. Cryoprotectant was used at a 20:1 molar ratio (cryoprotectant:lipids), preferably 10:1 and most preferably 5:1 to 1:1 with respect to the lipids emulsion. The sample was pre freezed for few hours and lyophilized for 24-120 hours using a appropriate freeze dryer with operational condenser temperature of −85 degree Celsius to −40 degree Celsius. The lyophilized material is stored at 5±3 degree Celsius or −20 degree Celsius.

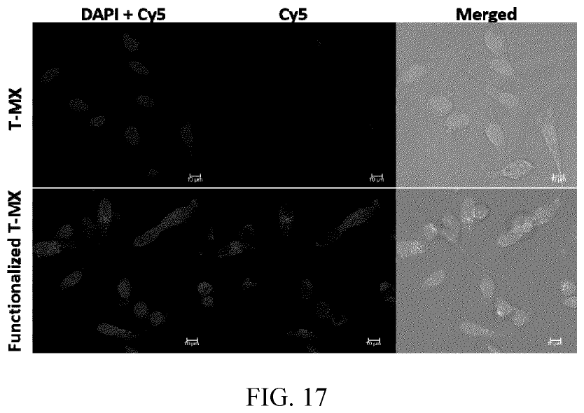

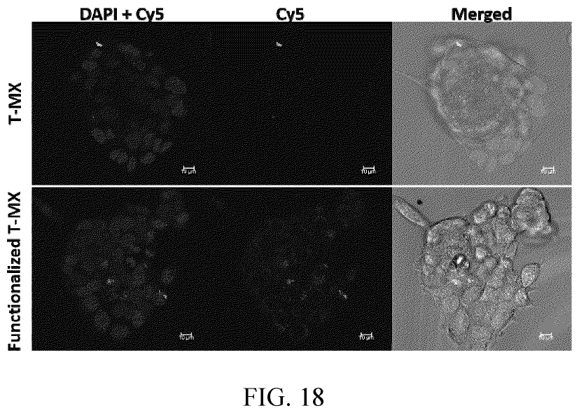

Targetability was evaluated using fluorescent T-MX. Fluorescent dye (Cy 5.5) labeled TERP (with and without Glu-urea-Lys) was used to synthesize fluorescent T-MX and PSMA targeting T-MX. Concentrations of these were then incubated with PC3 and DU145 prostate cancer cell lines for up to 24 hours and cell uptake monitored using a confocal laser scanning microscope. The cell uptake of PSMA ligand functionalized T-MX was compared with non-targeted T-MX for these two different cell lines (FIGS. 17 and 18). In both cases, the targeting T-MX showed higher uptake into cells than the non-targeting T-MX.

### Example 18

### Performance of T-MX in an Orthotopic GBM Mouse Model

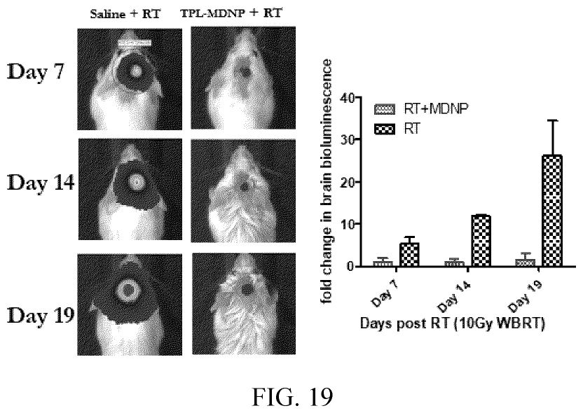

The radiation sensitization effect of T-MX was confirmed in vitro in human U87 GBM cells via colonagenic assay, following which, in vivo RT enhancement was assessed in an orthotopic GBM model (see FIG. 19). Combined T-MX+RT treatment significantly increased in vitro radiation sensitivity of U87 cells under hypoxia, and inhibited in vivo tumor progression by 13-fold when comparison to RT-only group in U87 brain tumor-bearing mice. Tumor growth is observed using bioluminescence imaging (BLI). Animal received a single dose of whole brain RT (10 Gy) alone, or in combination of T-MX treatment at 2 hr post NPs injection. Graphs showing the fold increase in tumor BLI signal compared to pre-RT, significant reduction of tumor growth for T-MX+RT at 7 days post treatment when compared to RT alone. The tumor growth was confirmed using the bioluminescence signal of U87-Luc cells.

1. A method of manufacture of a bioinorganic multifunctional theranostic nanoconstruct, the method comprising:
a. Adding manganese dioxide to a biocompatible matrix, thereby producing an emulsion of MnO₂ nanoparticles coated with the matrix;
b. Adding an additional layer to the emulsion; and
c. Processing the emulsion through a high-pressure homogenizer,
thereby manufacturing the bioinorganic multifunctional theranostic nanoconstruct.

2. The method of claim 1 further comprising concentrating the nanoconstruct.

3. The method of claim 2 in which the concentrating is performed using tangential flow filtration.

4. The method of claim 1 further comprising lyophilizing the concentrated nanoconstruct.

5. The method of claim 4 in which the lyophilizing takes place in the presence of a cryoprotectant.

6. The method of claim 1 in which the added manganese dioxide are precursor nanoparticles approximately 5 nanometers to approximately 80 nanometers in size.

7. The method of claim 1 in which the biocompatible matrix is lipid.

8. The method of claim 1 in which the biocompatible matrix is polymeric.

9. The method of claim 1 in which the biocompatible matrix is selected from polyelectrolyte-lipid, polyvinyl alcohol/lipid complex, graft TERP, phospholipids, poly(methacrylic acid)-polysorbate 80-starch (TERP), and fatty acids, optionally also including cholesterol.

10. The method of claim 1 in which the additional layer comprises TERP.

11. A bioinorganic multifunctional theranostic nanoconstruct comprising nanoscale manganese dioxide nanoparticles situated in a lipid emulsion, where the nanoparticles are coated with the lipid and further coated with a TERP functionalized with a targeting agent for cancer therapy.

12. The nanoconstruct of claim 10 in which the therapeutic agent targets PSMA and the functionalized TERP is TERP-Glu-urea-Lys.

13. A bioinorganic multifunctional theranostic nanoconstruct comprising nanoscale manganese dioxide emulsified with lipid and further coated with TERP, wherein the nanoconstruct is loaded with a magnetically resonant material for use in MRI imaging.

14. The nanoconstruct of claim 13 in which the magnetically resonant material is selected from gadolinium, manganese, iron, and oxides thereof.

15. A method of enhancing radiation therapy in a subject having a cancerous tumor, the method comprising, shortly prior to receiving radiation therapy, injecting the subject with a nanoconstruct of any of the preceding claims.

16. The method of claim 15 where the cancerous tumor is prostate cancer.

17. The method of claim 15 where the cancerous tumor is glioblastoma.

18. The method of claim 15 where the cancerous tumor is pancreatic cancer.

19. The method of claim 15 where the cancerous tumor is breast cancer.
